(19) United States
(12) Patent Application Publication
(10) Pub. No.: US 2009/0259204 A1
(43) Pub. Date: Oct. 15, 2009

(54) Method and Device for Applying Eye Drops

(76) Inventors: Shabty Galdeti, Rishon Letzion (IL); Uzi Ezra Havdsha, Tel Aviv (IL)

Correspondence Address: UZI EZRA HAVOSHA & PARTNERS, HADAR-DAFNA HOUSE 39 SHAOUL HAMELECH STREET, TEL AVIV, 64928, IL

(21) Appl. No.: 12/102,055

(22) Filed: Apr. 14, 2008

(51) Int. Cl. A61F 9/00; B65D 1/08; A61M 35/00

(52) U.S. Cl. 604/302; 604/295; 222/173

(57) ABSTRACT

An eye dropper with spring loaded soft tipped arms that hold open the eyelid to facilitate applying fluid eye medication drops to the eye. Eye dropper has attachment device for a bottle of liquid with a means of controllably dropping liquids into the open eye. Adjustable protrusions set maximum opening of eye and concurrently squeeze the bottle to controllably extract the eye drops.

### FIELD OF THE INVENTION

This invention is in the field of medical apparatus and in particular apparatus for applying fluids to eyes by holding the eyelids open a pre-set amount and dropping the required number of drops into the eye.

### BACKGROUND OF THE INVENTION

It is well known that opticians, eye doctors and individuals have difficulty applying drops to open eyes. The instinct of a person is to close the eye lids when a foreign body is about to approach or enter the eye. This eyelid movement is known as a reflex reaction. The main movement is done by the upper eyelid as is lowers to meet the lower eyelid. The patient often finds it difficult or even impossible to open his eye when he knows a foreign body will enter his eye, even when he knows that the foreign body is a liquid. The person applying the drops or the patient has to manually hold the eye open in order to apply the drops.

This problem is more acute when the patient is a child as children, generally, are more sensitive to such treatment.

### SUMMARY OF THE INVENTION

It is to be understood that both the foregoing general description and the following detailed description present embodiments of the invention and are intended to provide an overview or framework for understanding the nature and character of the invention as it is claimed.

This invention includes apparatus that performs the dual operation of opening the eye lids and holding a bottle of liquid above the eye to facilitate the application of the liquid to the eye. Such application is usually by pressing the bottle or the holed teat to cause one drop at a time to exit the hole in the teat. In a preferred embodiment of this invention adjustable protrusions on handles of the device press against the bottle of drops to controllably dispense the drops into the eye.

The eyelids are sensitive membranes that cover and protect the eyeball. The skin over the upper eyelids is one of the thinnest skins of the body. This invention takes these facts into account and provides for appropriately soft material like, for example, soft silicon pads to cover the endings of the arms that hold open the eyelids before and during the application of the drops.

Even though the top eyelid is the main lid that is held open when applying drops to the eye, the apparatus of this invention also holds open the lower eyelid. The lower eyelid does not move with a muscle movement like the upper eyelid but when held open a space is revealed between the lower portion of the eyeball and the inner surface of the lower eyelid. This space acts like a vessel that catches excess liquid drops applied to the eyeball and enables a larger surface of the eyeball to be in contact with the said liquid drops.

The apparatus of this invention comprises two arms where each arm has at one end a covering of a soft pad that is suitable material for contact with the eyelid. At the distal end of each arm is a handle that is pressed inward, relative to the center point of the bottle on the bottle's longitudinal axis, pivoted at the connection to the bottle. The pressing of the handles inward causes the soft pads move in an outward movement, relative to the same center point, because of the pivotal effect. This outward movement is the movement that opens the eye lids and holds the eyelids open.

Both arms are pivotally attached with a spring mechanism to the eye drops bottle or bottle holder so that the inward pressing action at the handle end of the arm causes an outward movement of the soft pads at the other end of the arm.

When the arm handles are not pressed the spring mechanism keeps the handles at their widest apart and the soft pads closest together. This enables the greatest degree of control when pressing the arm handles thereby separating the soft pads, which movement opens the eyelids.

On each handle is a sliding adjustable protruding piece to pre-set the maximum distance moveable by the soft pads and therefore the maximum opening of the eyelids. These protruding pieces are also a safety device to prevent accidental excess opening of the eyelids. The adjustment is also to cater for the different sized eyes of adults and children. Children would require a smaller opening of the eye and so the adjustable protruding piece would be moved towards the open end of the bottle thereby enabling the soft pads to open the minimum amount.

The eye drop apparatus of this invention, in the preferred embodiment, is an integral part of eye drop bottle. The eye drop bottle could be manufactured with the soft pads and handles ready attached to the bottle. Each bottle packed and dispensed with its eye opening equipment attached and ready to use. Alternatively, the eye drop bottle could be a re-useable bottle or a device to hold standard size eye drop bottles or an adjustable device to hold different sized bottles.

### BRIEF DESCRIPTION OF THE DRAWINGS

The accompanying drawings are included to provide a further understanding of the invention and are incorporated into and constitute a part of this specification. The drawings illustrate various embodiments of the invention and, together with the description, serve to explain the principles and operations of the invention, by way of example only and not to limit the invention to these descriptions only.

FIG. 1 is a schematic depiction of the eyelid opener and dropper of this invention.

FIG. 2 is a schematic depiction of the eyelid opener and dropper of this invention being applied on a user.

FIG. 3 is a schematic depiction of the eyelid opener and dropper with a screwing mechanism.

FIG. 4 is a schematic depiction of the eyelid opener and dropper with a screwing mechanism and a built in lid.

FIG. 5 is a schematic depiction of an upper eye lid soft pad.

### DETAILED DESCRIPTION OF THE PREFERRED EMBODIMENTS

As will be appreciated the present invention is capable of other and different embodiments than those discussed above and described in more detail below, and its several details are capable of modifications in various aspects, all without departing from the spirit of the invention. Accordingly, the drawings and description of the embodiments set forth below are to be regarded as illustrative in nature and not restrictive.

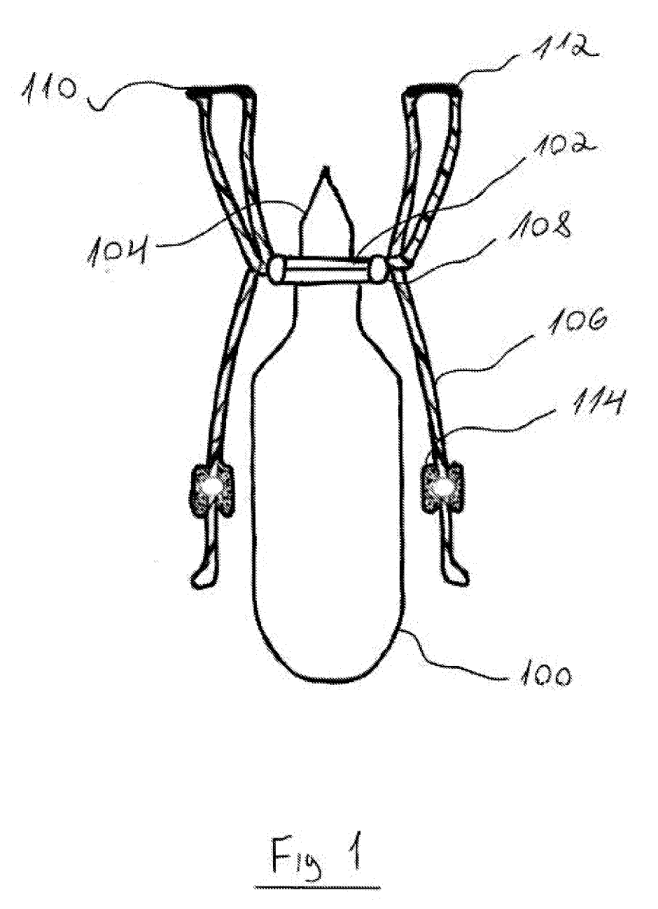

FIG. 1 shows the bottle 100 that holds the fluid eye medication drops. The bottle has a neck that fits into a sleeve 102. The sleeve 102 would be adjustable in circumference to fit different sized bottles. The fitting of the bottle in the sleeve 102 is tight so that the bottle 100 is held safely and steadily in place to ensure accurate positioning of the teat 104 over the eye.

The handles 106 are pivoted at the point where they are attached to the springs 108. The springs 108 are attached to the sleeve 102. The springs 108 could be attached directly to the bottle 100 in the area of the bottle neck. At the distal end of the handles 106 are the soft pads. The soft pad 110 is for the upper eye-lid and is shaped to match approximately the shape of the upper lid. The upper-eye lid soft pad is used to hold open the upper lid of the eye. The lower eye lid is held a distance from the eye ball surface with the aid of the soft pad 112. The soft pads 110 and 112 could be made of any soft material that is suitable for contact with the delicate lid of the eye for example, soft silicon.

The sliding adjustable protruding piece 114 is for setting the maximum width between the soft pads 110 and 112. This is also a safety device preventing the pads 110 and 112 from distancing one from the other farther than desirable. The protrusions 114 work by controllably sliding in a groove in the handle 106. The protrusion 114 protrudes from the handle 106 in the direction of the bottle 100. When the protrusion 114 is closer to the springs 108 the protrusion 114 prevents the handles 106 from being opened more than a given amount as the protrusion 114 comes in contact with the sides of the bottle 100.

In one embodiment the protrusions 114 coming in contact with the bottle 100 could a method of extracting drops from the bottle 100 where the sides of the bottle are flexible. The same movement of holding the eye lids open the required amount, causes the protrusions 114 to press against the side of the bottle 100 thereby causing at least one drop of liquid to be released from the teat 104. This embodiment would enable the user to open the eyelids of the patient and controllably and simultaneously apply the drops with one hand, leaving the other hand free for other activity.

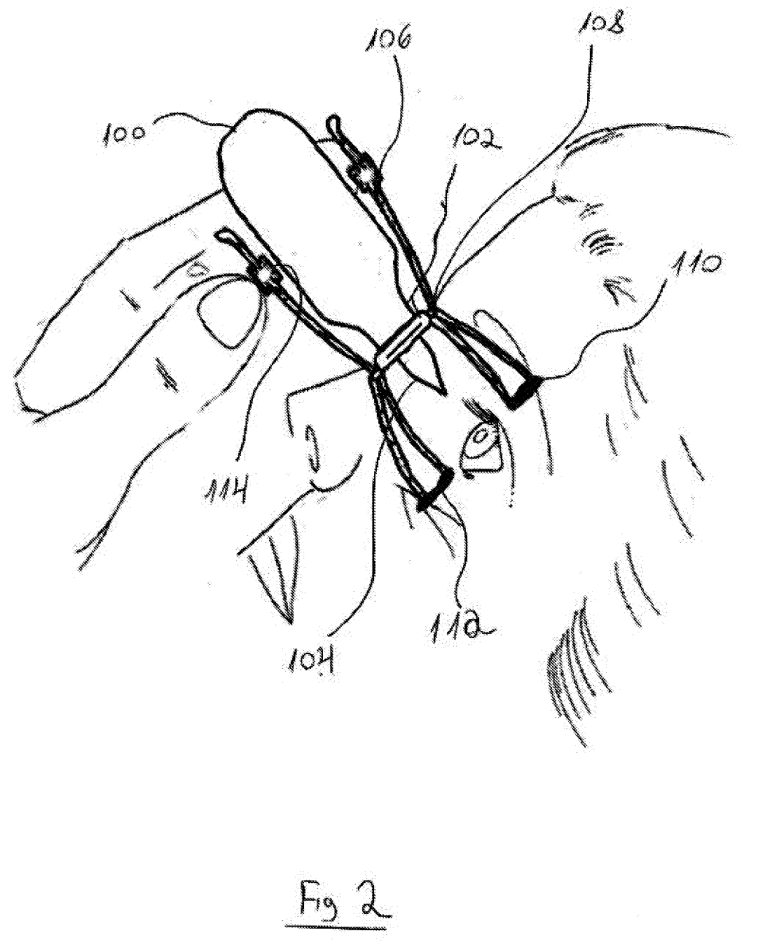

FIG. 2 shows the eyelid opener and dropper of this invention as being explained in FIG. 1 with the illustration of how the eyelid opener and dropper is being applied on a user.

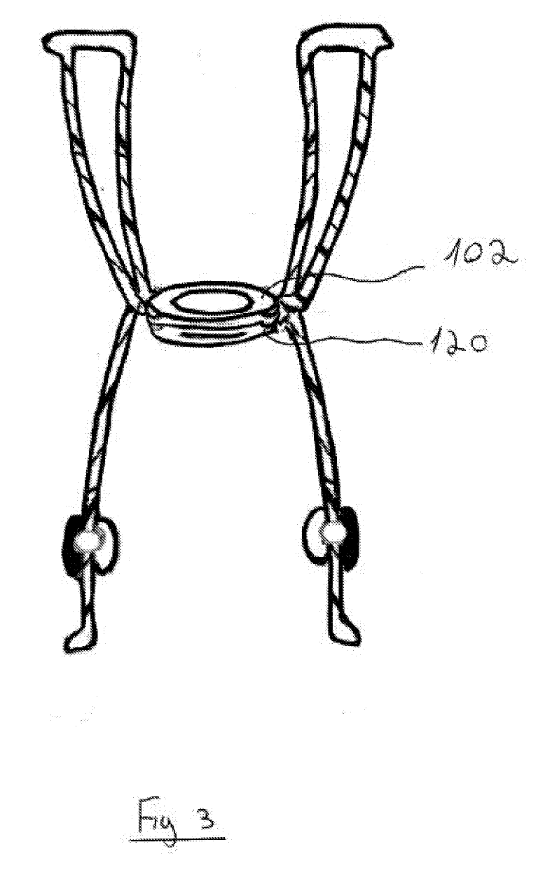

FIG. 3 shows an eyelid opener and dropper with a screwing mechanism. The neck of a battle that holds the fluid eye medication drops is screwed into a sleeve 102 by a screwing mechanism 120. The screwing of the bottle in the sleeve 102 enable the bottle to be held safely and steadily in place to ensure accurate positioning of the teat over the eye.

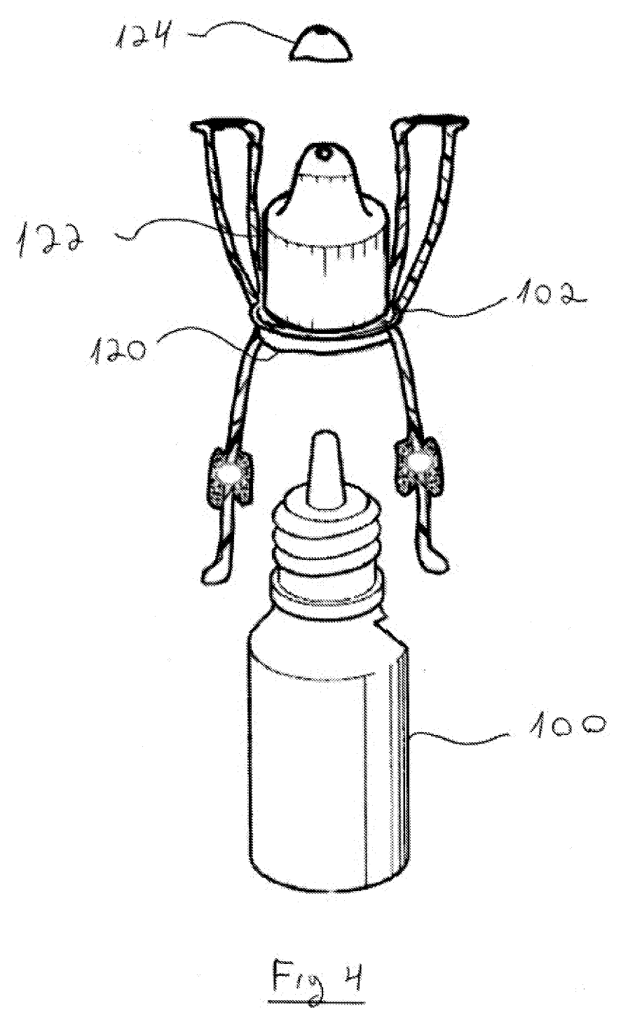

FIG. 4 shows an eyelid opener and dropper with a built in lid. The battle 100 that holds the fluid eye medication drops is screwed into a sleeve 102 by a screwing mechanism 120. The built in lid 122 with a small portion 124 on top of the lid 122 that is removable, enable the user to screw the battle 100 to the eyelid opener and dropper only before the first use and leave the battle attached to the eyelid opener and dropper until the eye drops are finished or the user decide otherwise. The lid 122 is covering the teat 104 keeping it clean and hygienic for safe use. Before every use the user only needs to remove the removable portion 124 from the lid revealing the teat 104 of the battle 100, and the eyelid opener and dropper is ready to be used.

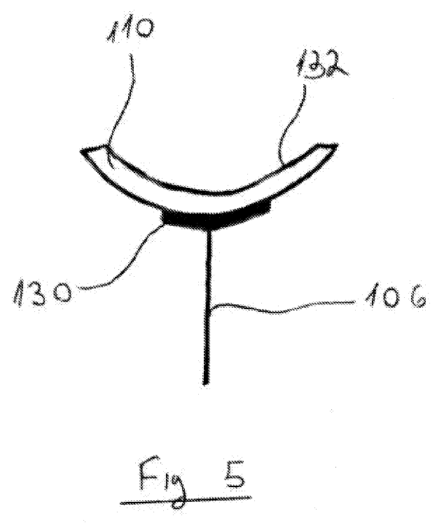

FIG. 5 shows a soft pad 110 from the cross sectional side view. The pad 110 is attached to the arm 106 on the attachment plate 130. The pad 110 is shaped with a curvature 132 that will fit the curvature of the upper eye lid. There could be different sized soft pads for adults and children.

What is claimed: The present invention is not intended to be limited to the embodiments described above, but to encompass any and all embodiments within the scope of the following claims.

1. A bottle for applying fluid eye medication comprising;
a. two springs attached to opposite sides of the said bottle on a diametric axis of the said bottle,
b. an arm pivotally attached to each of the said springs, the said two arms being substantially in the same plane as the said bottle,
c. a soft pad attached to the end of each said arm, at the end closest to the said bottle,
d. a handle at the distal end of each said arm incorporating a groove facing the said bottle where the said groove runs parallel to the length of the said handle, in which a protruding piece may be moved up and down in the said groove, and
e. a controllably adjustable protruding piece housed partially in the said groove of each said handle and partially protruding from each said handle in the direction of the said bottle whereby the said pads are moved away from each other and the said protruding pieces may press against the sides of the bottle by the action of the said handles being moved towards each other.

2. A holder and eye dropper device comprising;
a. a substantially round shaped adjustable bottle holder,
b. two springs attached to opposite sides of the said bottle holder on a diametric axis of the said bottle holder,
c. an arm pivotally attached to each of the said springs, being substantially in the same plane as the said bottle when inserted in the said bottle holder,
d. a handle at the one end of each said arm incorporating a groove facing the said bottle where the said groove runs parallel to the length of the said handle, in which a protruding piece may be moved up and down in the said groove,
e. a controllably adjustable protruding piece housed partially in the said groove of each said handle and partially protruding from each said handle in the direction of the said bottle, and
f. a soft pad attached to the distal end of each said arm whereby the said pads are moved away from each other and the said protruding pieces may press against the sides of the said bottle by the action of the said handles being moved towards each other.

3. An eye dropper device for applying fluid eye medication to eyes comprising the following steps;
a. placing one soft pad on the upper lid of an eye,
b. placing the other soft pad on the lower lid of the said eye,
c. adjusting protruding pieces in the grooves of each of the two pivoted arm handles to pre-set the maximum distance between the said pads,
d. pressing the said handles towards each other in order to separate the said pads thereby opening the said lids of the said eye,
e. squeezing the said bottle to cause at least one drop of liquid out of the said bottle and onto the said eye.

4. A bottle for applying fluid eye medication as claimed in claim 1 wherein the said bottle has flexible sides thereby enabling at least one drop to be squeezed from the said bottle when the said protruding pieces are pressed against the said sides.

5. An eye dropper device for applying fluid eye medication to eyes as claimed in claim 3 further comprising the step of adjusting the said protruding piece in a groove in the said handle so that when the said protruding piece presses the sides of the said bottle at least one drop is squeezed out of the teat of the said bottle and simultaneously the said pads reach their desired distance apart from each other.

6. A holder and eye dropper as claimed in claim 2 wherein the said bottle holder is adjustable to accommodate different sized bottles.

7. A holder and eye dropper as claimed in claim 2 wherein the said bottle holder has a screw mechanism enabling a battle that holds the fluid eye medication drops to be screwed in said bottle holder.

8. A holder and eye dropper as claimed in claim 2 wherein the said bottle holder has a built in lid. Said built in lid further comprise a small, removable portion on top of said lid.

9. A holder and eye dropper as claimed in claim 7 wherein the said bottle holder has a built in lid. Said built in lid further comprise a small, removable portion on top of said lid.

10. A bottle for applying fluid eye medication as claimed in claim 1 wherein one said soft pad is shaped substantially to fit the shape of an upper eye lid.

11. A holder for an eye dropper as claimed in claim 2 wherein one said soft pad is shaped substantially to fit the shape of an upper eye lid.

12. A bottle for applying fluid eye medication as claimed in claim 1 wherein the said soft pads are made of one or more of the following list namely, silicon, soft silicon, padded cotton, padded material and inflated rubber.

13. A holder for an eye dropper as claimed in claim 2 wherein the said soft pads are made of one or more of the following list namely, silicon, soft silicon, padded cotton, padded material and inflated rubber.

14. A holder for an eye dropper as claimed in claim 2 further comprising a re-usable bottle that is removeably attached to the said bottle holder.
